(19) United States
(12) Patent Application Publication
(10) Pub. No.: US 2014/0065712 A1
(43) Pub. Date: Mar. 6, 2014

(54) RAPID DETECTION OF UREA IN ADULTERATED MILK USING THREAD BASED MICROFLUIDIC SYSTEM

(71) Applicants: Jayant KHANDARE, Mumbai (IN); Debjani Paul, Mumbai (IN); Shashwat Banerjee, Mumbai (IN); Neetika Taneja, Mumbai (IN); Mahesh Shidore, (US)

(72) Inventors: Jayant KHANDARE, Mumbai (IN); Debjani Paul, Mumbai (IN); Shashwat Banerjee, Mumbai (IN); Neetika Taneja, Mumbai (IN); Mahesh Shidore, (US)

(73) Assignee: Piramal Enterprises Limited (PEL).

(21) Appl. No.: 13/596,518

(22) Filed: Aug. 28, 2012

(51) Int. Cl. G01N 33/62; G01N 21/78

(52) U.S. Cl. CPC G01N 33/62; G01N 21/78
USPC 436/23; 422/400; 422/420

(57) ABSTRACT

A system for detecting an adulterant in a sample of milk. One or more drops of milk are added to a first location of a system and the presence or absence of the adulterant is detected in a second location of the system using a material treated with a detection reagent.

### FIELD OF THE INVENTION

The present invention is directed to a microfluidic system that can be used to detect adulterants in a liquid.

### BACKGROUND OF THE INVENTION

Unlike conventional microfluidic systems that require an external pumping mechanism, most liquids can easily wick through thread by capillary action. Until now, thread based microfluidics systems have been in the context of low-cost diagnostics to detect clinically relevant substances such as, glucose, bovine serum albumin, uric acid, nitrate, alkaline phosphatase, etc. [1-4] or for blood typing.

Inexpensive, user-friendly, portable, and easily disposable microfluidic systems are desired for monitoring and detecting adulterants in consumables.

### SUMMARY OF THE INVENTION

This invention relates to a cotton thread based microfluidic system to detect adulterants in milk.

Another aspect of the invention is that the cotton thread-based microfluidic system is in the form of a chip.

A further aspect of the invention is a cotton thread-based microfluidic system to detect urea in milk.

Another aspect of the invention is a cotton thread-based microfluidic system that can be used to detect adulterants other than urea in milk.

Another aspect of the invention is a cotton thread-based microfluidic system to detect urea and other adulterants in milk.

Still another aspect of the invention is a method for detecting an adulterant in milk.

Still another aspect of the invention is a method for detecting urea in milk.

Another aspect of the invention is a method for detecting urea and one or more other adulterants in milk.

### BRIEF DESCRIPTION OF THE FIGURES

FIG. 1 shows the top and bottom of a microfluidic chip.

FIG. 2 shows the components of a microfluidic chip of the invention.

FIG. 3A shows a microfluidic chip that can be used to detect urea.

FIG. 3B shows a multichannel microfluidic chip that can be used to detect urea and at least one adulterant.

FIG. 4 shows a graph illustrating the concentration of urea detected in milk and a color scale used to identify the concentration of urea in milk.

FIG. 5 shows a multichannel system for detection of urea, glucose and caustic soda (NaOH).

### DETAILED DESCRIPTION OF THE INVENTION

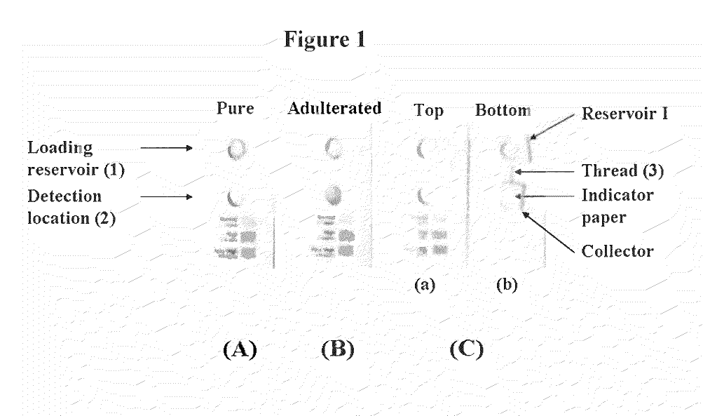

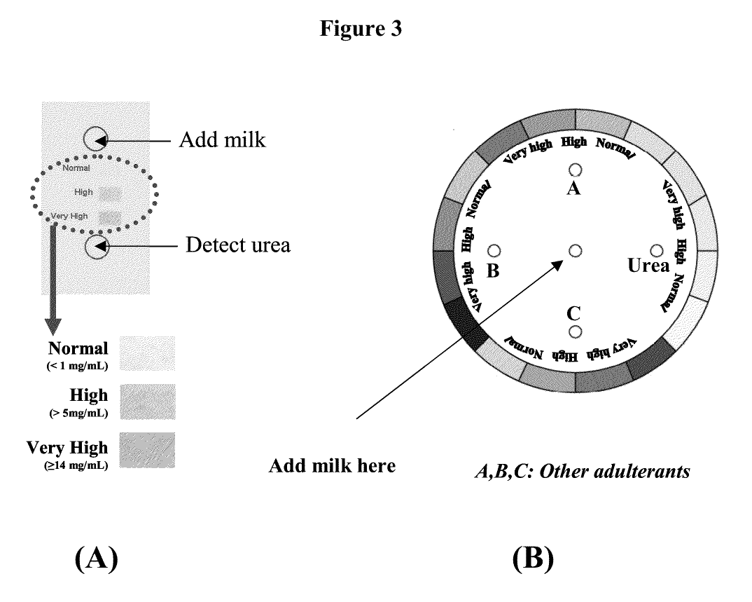

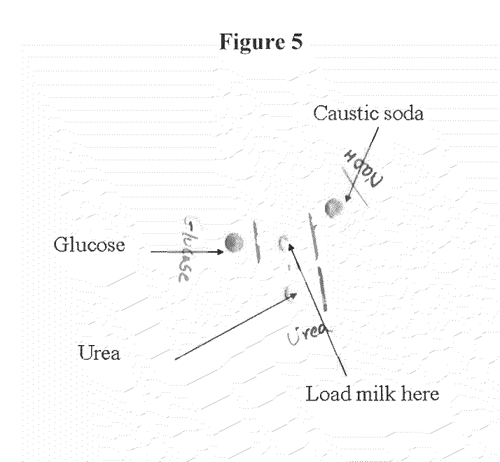

FIGS. 1, 2, 3 and 5 show microfluidic chips which are used to detect adulterants in milk. FIGS. 3B and 5 show multichannel microfluidic chips that can be used to detect the presence of two or more adulterants in a sample of milk.

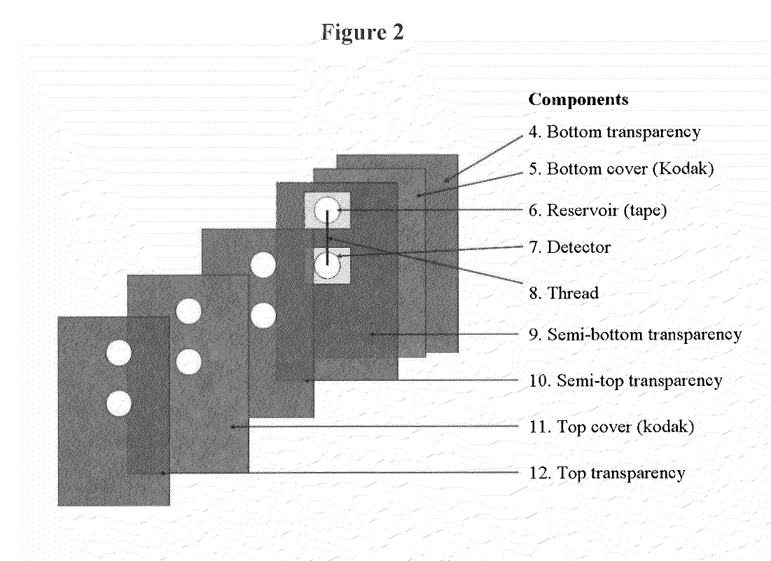

FIG. 1C shows the top (a) and bottom (b) of a microfluidic chip. The top of the chip system comprises a loading reservoir 1 and a detection location 2. The back of loading reservoir 1 and a detection location 2 are shown in FIG. 1C (b). The reservoir and detection location are connected by at least one cotton thread. The detection location includes an area in which a material that is treated with a detection reagent can be loaded. The material treated with a detection reagent for detecting an adulterant can be inserted in the detection location just prior to use. In some aspects of the invention, the material that is treated with a detection reagent is pre-loaded. In an aspect of the invention, illustrated in FIG. 2 a microfluidic chip comprises an area for loading a test sample (loading reservoir 6) and a second area that is a detection location 7 for detecting the presence or absence of an adulterant. Detection locations shown in FIGS. 1 and 2 may have two punched double sided adhesive tapes into which the material treated with a detection reagent is placed.

In an aspect of the invention, the microfluidic chip shown in FIG. 2 includes a bottom transparency 4, a bottom cover 5 wherein the bottom transparency covers the bottom cover that includes a loading reservoir 5 made from punched tape, a thread 7 that is attached to loading reservoir 6 and detection location 7 by the reservoir tapes. This is covered by a semi-bottom transparency 9, semi-top transparency 10, top cover 11 and top transparency 12. Holes are punched in the semi-top transparency 10, top cover 11 and top transparency 12. In an aspect of the invention, double sided adhesive tape is placed at the detection location. The double sided tape with a hole punched in it acts as the reservoir. The material that is treated with a detection reagent for detecting the presence of an adulterant is placed inside the detection reservoir. In an aspect of the invention, when the material is greater in size than detection location, it is not necessary to use an additional component to fix the material inside the detection location.

In an aspect of the invention, the material that will be used as the indicator medium is first treated with alginate gel, dried and treated with a detection reagent. The material is then dried and inserted into the detection reservoir of the chip.

Whatman paper is an example of a material that can be used as the indicator medium. Whatman paper is treated with a detection reagent and inserted in a detection location. Whatman paper can be treated with urea indicator or another chemical that can be used for detection of another adulterant in milk. Ehrlich reagent can be used as a urea indicator.

Other methods for detecting urea using this microfluidic chip can also be used. These include using reagents other than Ehrlich's reagent for detecting the presence of urea or other adulterants.

Non-limiting examples of materials that can be used to make the chip are paper, tape, plastic transparencies, plastic films, or the like or a combination thereof can be used to make the chip. Non-limiting examples of materials that can be treated with the detection reagent are paper, filter paper, polymeric material and the like.

Cotton thread is used as the substrate between the loading reservoir and the detection location. The cotton thread functions as a pre-defined microfluidic channel. The high aspect ratio of cotton thread keeps the liquid flowing through it confined to one dimension without requiring any expensive surface patterning (e.g. lithography). In the microfluidic system of the invention, cotton thread is used as an appropriate substrate for transporting milk. Other threads (for example, silk thread) are not suitable transporters for milk, since their composition could interfere with milk proteins. In an aspect of the invention illustrated in FIG. 1C (b), one end of the cotton thread is attached to the sample loading reservoir and the other end is attached to the detection location. Another example is shown in FIG. 2.

Use of multiple threads and a shorter channel length leads to faster detection by decreasing resistance to milk flow in the channel. The length of the thread is based on the dimensions of the chip and the distance separating the loading reservoir and detection location. Two or more cotton threads can be braided together to form a liquid-transporting channel. The inter-thread gaps formed by braiding are larger than the intra-thread gaps present in a single thread. The inter-thread gaps aid the fluid flow by capillary action. In an aspect of the invention three cotton threads are braided together.

A cotton thread microfluidic chip described herein can be used for the detection of urea in adulterated milk and artificial or synthetic milk. Chips of this type can also be used to detect other adulterants in milk. The number of adulterants that can be detected in a sample is based on the number of detection locations. The chip can also include a color scale as shown in FIGS. 1 and 3 on the front of the chip to aid in identifying the amount of adulterant present in the milk sample. If the chip is used to detect two or more adulterants multiple color scales can be used. An example of this is shown in FIG. 3B.

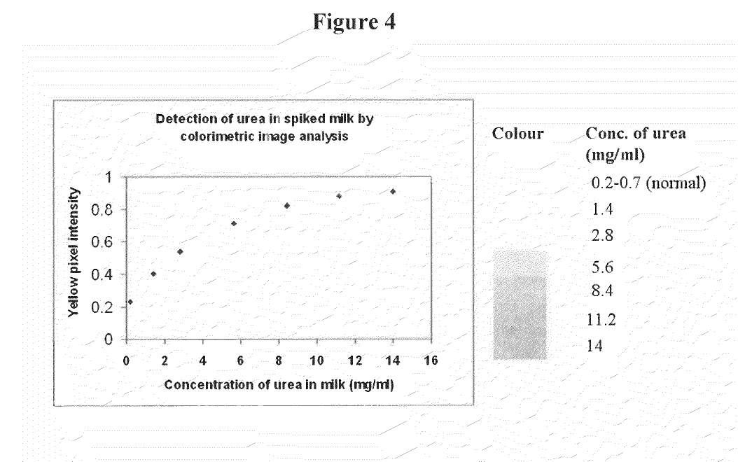

After loading one or more drops of milk, adulterated milk or adulterated artificial or synthetic milk or a combination thereof in the loading reservoir of the microfluidic chip (See FIG. 1), the presence of urea can be detected within 10-60 seconds. The yellow color on the chip indicates the presence of urea in range from 1.5 mg/mL to 14 mg/mL, and above. The yellow color scale is used to determine the amount of urea in a sample. The amount of urea is determined by using as analytical method and/or by image analysis (using Image J). The Image J images represent the pixel intensities (as yellow color) based on the amount of urea in the sample (FIG. 4). FIG. 1A shows that the sample did not contain urea. The presence of urea was detected as shown in FIG. 1B. Other methods for detecting the amount of urea can be used.

A microfluidic chip system can include multiple channels in order to detect two or more adulterants in milk. Examples of these type of chips are shown in FIGS. 3B and 5. Non-limiting examples of other adulterants that can be detected are peroxide (H₂O₂), sodium hydroxide, potassium hydroxide, bleaching agents, salicylic acid, glucose, sodium chloride etc. Non-limiting examples of reagents that can be used to identify adulterants are:

| Sr. No | Adulterants | Reagents |
|---|---|---|
| 1 | Glucose | Glucose oxidase and horseradish |
| | | peroxidase reagent (GOP) |
| 2 | Caustic soda (NaOH) | Phenolphthalein |
| 3 | KOH | Phenolphthalein |
| 3 | Proteins | Bromophenol blue |

The time for detecting the presence of an adulterant depends on the adulterant and the reagent. Cotton thread based microfluidic chip can detect urea in adulterated milk or adulterated artificial or synthetic milk in less than five (5) minutes. Urea can be detected within 5 to 120 seconds or even in 10-60 seconds.

An advantage of the invention is that the cotton thread based microfluidic chip is inexpensive and simple to fabricate.

As used in this specification and the appended claims, milk means milk obtained from an animal or a plant, artificial or synthetic milk or a combination thereof unless the context clearly dictates otherwise.

As used in this specification and the appended claims, singular forms “a,” “an,” and “the” include plural referents unless the content clearly dictates otherwise. It should also be noted that the term “or” is generally employed in its sense including “and/or” unless the content clearly dictates otherwise.

Unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

It is to be understood that the invention may assume various alternative variations and step sequences, except where expressly specified to the contrary.

The invention is further understood by reference to the following Examples, which are intended to be purely exemplary of the invention and are not intended to limit the scope of the invention.

### Example 1

### 1. Microfluidics

Cotton (TV 10/P2 from Rainbow, India) thread, double-sided foam tape (Microfoam, India) and polyester overhead transparency sheets (Desmat Office Products, India) were sourced from the local suppliers in India. All threads were used without any further chemical treatment.

### 2. Determination by Ehrlich Reagent

Urea in adulterated milk was identified by using Ehrlich reagent.

### 3. Chemicals

4-Dimethylamino benzaldehyde was obtained from Arro Biochem (India). All other chemicals were of analytical grade unless specified otherwise.

### 4. Reagents

Alcoholic Ehrlich Reagent:

4-Dimethylamino benzaldehyde, 3.2 g., dissolved into 90 ml absolute ethanol. To this solution 10 ml of HCl (35%) was added. The same solution can also be prepared by dissolving 3.2 g of 4-Dimethylamino benzaldehyde in 95 mL of absolute ethanol and then adding 5 mL of conc. HNO₃. Other acids e.g. H₂SO₄, H₃PO₄, etc. can also be used.

### 5. Alginate Gel

Sodium alginate is dispersed in deionised water (3% w/v) for 12 hours with constant stirring. The alginate gel thus formed is used for coating the indicator paper which is further dipped in the indicator solution (Alcoholic Ehrlich Reagent).

### 6. Composition of Synthetic Milk

Each 10 ml contains

| | | |
|---|---|---|
| No | Ingredient | Quantity |

| | | | |
|---|---|---|---|
| 1 | Refined oil | 1 | ml |
| 2 | Span-20 | 1 | ml |
| 3 | LabrafacWL1349 | 200 | ul |
| 4 | 0.1N NaOH | 50 | ul |
| 5 | Urea | 140 | mg |

| | | |
|---|---|---|
| 6 | Water | q.s to 10 ml |

Procedure:
1. Mix Refined oil, Span −20 and Labrafac WL 1349.
2. Add given quantity of 0.1 NaOH to form a thick primary emulsion
3. Add 5 ml of water with continuous stirring.
4. Dissolve urea in 1 ml of hot water and add it to the above prepared emulsion.
5. Make up the volume up to 10 ml with water by stirring.

### Example 2

### Procedure for Preparation of Urea Detection Indicator

1. The Whatman's paper is first dipped in alginate gel (2-3% w/v) and dried with hot air blower. This alginate treated paper is then dipped in the alcoholic Ehrlich reagent and dried once again. Holes are punched from the treated paper to obtain discs that are about 3 mm in diameter and inserted into the detection location of the chip (FIG. 1A).

### REFERENCES

1. X. Li, J. Tian and W. Shen, Applied Materials and Interfaces, 2, 1 (2010).
2. M. Reches, K. A. Mirica, R. Dasgupta, M. D. Dickey, M. J. Butte and G. M. Whitesides, Applied Materials and Interfaces, 2, 1722 (2010).
3. D. R. Ballerini, X. Li and W. Shen, Biomicrofluidics 5, 014105 (2011).
4. Roozbeh Safavieh, Gina Z. Zhou and David Juncker, Lab on a Chip, 11, 2618 (2011).
5. D. R. Ballerini, X. Li and Wei Shen, Anal Bioanal Chem 399, 1869 (2011).
6. S. Banerjee, J. Khandare, A. Roychowdhury, R. Janrao and D. Paul (submitted).

1. A system comprising a first location, a second location and at least one cotton thread attached at a first end to the first location and at a second end to the second location.

2. The system according to claim 1, wherein the first location is a reservoir.

3. The system according to claim 1, wherein the second location is used for detection.

4. The system according to claim 1, wherein a plurality of cotton threads are used.

5. The system according to claim 4, wherein the threads are braided.

6. The system according to claim 3, wherein the second location comprises a material treated with a detection reagent.

7. The system according to claim 6, wherein the detection reagent is Ehrlich's reagent.

8. The system according to claim 6, wherein the material is paper.

9. A method of detecting an adulterant in a sample of milk comprising the steps of loading one or more drops of milk in a first location of the system of claim 1 and detecting the presence of the adulterant in the second location of the system of claim 1 wherein a material treated with a detection reagent is present in the second location.

10. The method of claim 9, wherein the material is treated with a polymer gel and dried before being treated with a detection reagent.

11. The method of claim 9, wherein the milk is synthetic milk.

12. The method of claim 9, wherein the material is paper.

13. The method of claim 9, wherein the adulterant is urea.

14. The method of claim 9, wherein the detecting reagent is Ehrlich's reagent.

15. The method of claim 9, wherein the adulterant is one or more of urea, glucose, sodium hydroxide (NaOH), potassium hydroxide (KOH), bleaching agents, and salicylic acid.
